SEQUENCE LISTING (10 sequences)

SEQ ID NO: 1 - protein, 206 aa, Homo sapiens
QTDMSRKAFVFPKESDTSYVSLKAPLTKPLKAFTVCLHFYTELSSTRGTVFSRMPPRDKTMRFFIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - protein, 206 aa, Homo sapiens
QTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 3 - protein, 227 aa, Artificial Sequence
  Synthetic Sequence
MKQSTIALALLPLLFTPVTKAQTDMSRKAFVFPKESDTSYVSLKAPLTKPLKAFTVCLHFYTELSSTRGTVFSRMPPRDKTMRFFIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 4 - protein, 227 aa, Artificial Sequence
  Synthetic Sequence
MKQSTIALALLPLLFTPVTKAQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 5 - DNA, 621 bp, Homo sapiens
cagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccggggtacagtattttctcgtatgccaccaagagacaagacaatgagattcttcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 6 - DNA, 621 bp, Homo sapiens
cagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 7 - DNA, 684 bp, Artificial Sequence
  Synthetic Sequence
atgaaacaaagcactattgcactggcactcttaccgttactgtttacccctgtgacaaaagcccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccggggtacagtattttctcgtatgccaccaagagacaagacaatgagattcttcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 8 - DNA, 684 bp, Artificial Sequence
  Synthetic Sequence
atgaaacaaagcactattgcactggcactcttaccgttactgtttacccctgtgacaaaagcccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 9 - DNA, 45 bp, Artificial Sequence
  Forward Primer for CRP Gene in Example 1
cacacaggaaacacatatgatgaaacaaagcactattgcactggc

SEQ ID NO: 10 - DNA, 34 bp, Artificial Sequence
  Reverse Primer for CRP Gene in Example 1
tctagaactagtggatcctcagggccacagctgg

(19) United States
(12) Patent Application Publication
(10) Pub. No.: US 2023/0039528 A1
(43) Pub. Date: Feb. 9, 2023

(54) RECOMBINANT C-REACTIVE PROTEIN

(71) Applicant: TOYOBO CO., LTD., Osaka (JP)

(72) Inventors: Ayaka MISHIMA, Tsuruga (JP); Yosuke SUMIDA, Osaka (JP)

(73) Assignee: TOYOBO CO., LTD., Osaka (JP)

(21) Appl. No.: 17/780,893

(22) PCT Filed: Oct. 23, 2020
(86) PCT No.: PCT/JP2020/039893

§ 371 Date: May 27, 2022

(30) Foreign Application Priority Data
Nov. 29, 2019 (JP) 2019-216784
Aug. 21, 2020 (JP) 2020-140434

(51) Int. Cl. G01N 33/539; C07K 14/47

(52) U.S. Cl. CPC G01N 33/539; C07K 14/4737; G01N 2333/4737

(57) ABSTRACT

The accuracy of immunoassay using a latex reagent is improved in a high CRP concentration range. Provided are C-reactive proteins generated by genetic recombination, 55% or more of the C-reactive proteins having a pyroglutamylated N-terminal.

### TECHNICAL FIELD

The present invention relates to C-reactive proteins (hereinafter also referred to as “CRPs”) produced by a genetic recombination technique, and use of the same. More specifically, the present invention relates to recombinant CRPs obtained by transforming the N-terminal structure of CRPs, a calibrator using the recombinant CRPs, control serum using the recombinant CRPs, and a method for quantifying CRPs based on an antibody-antigen reaction.

### BACKGROUND ART

CRP is a protein that exhibits a precipitation reaction with the C-polysaccharide in the pneumococcal capsule, and is also known as a typical inflammatory marker because it is a type of acute phase protein. The blood concentration of CRP increases remarkably in infectious diseases and inflammatory diseases, and decreases sharply with the recovery of symptoms. Accordingly, the quantification of CRP is used as an index to determine the severity of various diseases and to observe the course of treatment. The CRP concentration in the blood of healthy people is generally 0.3 mg/dL or less, whereas in patients with inflammation or inflammatory diseases, the CRP concentration rapidly increases hundreds to thousands of times in a short period of time. Therefore, in the measurement of CRP in a sample, it is required to accurately measure a wide range of CRP concentration from low concentration to high concentration.

As a method for quantifying blood CRP concentrations in the clinical laboratory field, there is a measurement method using an antigen-antibody reaction, and known methods are enzyme immunoassay, luminescent immunoassay, latex turbidimetric immunoassay, immunochromatography, and the like. In particular, among these measurement methods, latex turbidimetric immunoassay is widely used for daily inspections because it is easy to operate and the measurement can be automated by an analyzer. In latex turbidimetric immunoassay, the blood CRP concentration is quantified from a calibration curve using a calibrator with a known concentration. The accuracy of the calibration curve is ensured by measuring control serum.

Although natural CRP purified from human body fluids such as ascites fluid is used in the above-mentioned calibrator and control serum, there is a risk that serum components other than CRP may contaminate them and affect the measurement of blood CRP concentration. In addition, there is a risk of secondary pathogenic infection due to the handling of human body fluids as biological raw materials in the isolation and purification stages of CRP. There are also safety issues in production. Furthermore, the CRP content in human body fluids is variable, which causes a problem in terms of stable supply. On the other hand, recombinant CRP using microorganisms or the like does not use human body fluids as raw materials, and thus does not carry the risk of contamination with human-derived serum components or secondary infections. Accordingly, if a stable expression system for recombinant protein can be constructed using gene engineering technology, the desired protein can be stably supplied.

Regarding the expression of recombinant CRP by microorganisms, successful examples in Escherichia coli, yeast, etc., have already been reported (PTL 1 and NPL 1). However, the measurement of the recombinant CRP concentration by latex turbidimetric immunoassay had a problem that the measured values were lower than the actual CRP concentration in a high CRP concentration range. Therefore, in order to use recombinant CRP as a diagnostic raw material for a calibrator, control serum, etc., it is necessary to improve the accuracy of measurements in a high CRP concentration range.

Many proteins undergo chemical characterization or structural transformation by post-translational modifications. One of the post-translational modifications is pyroglutamylation in which the carboxyl group and amino group of glutamine or glutamine acid are converted into pyroglutamic acid by an intramolecular condensation reaction. Plants and animals have many pyroglutamyl peptides modified by pyroglutamylation, and there are proteins such as β-amyloid, collagen, and IgG₂. CRP is also a pyroglutamyl peptide. However, it has been reported that recombinant CRP includes those whose N-terminal is still glutamine, and those whose N-terminal is converted into pyroglutamic acid (NPL 2). There has been no report on the relationship between the N-terminal structure of CRP and the antibody-antigen reaction in latex turbidimetric immunoassay.

### CITATION LIST

### Patent Literature

PTL 1: JP2000-14388A

### Non-Patent Literature

NPL 1: Toshio Tanaka et al., Biochem. Biophys. Res. Commun., 295 (2002), pp. 163-166
NPL 2: Journal of Clinical Laboratory Medicine, Vol. 46, No. 9, pp. 973-981 (September 2002)

### SUMMARY OF INVENTION

### Technical Problem

An object of the present invention is to improve the accuracy of measurements in a high CRP concentration range particularly when using latex turbidimetric immunoassay.

### Solution to Problem

As a result of extensive research in view of the above circumstances, the present inventors found a way to solve the above problems by transforming the N-terminal structure of recombinant CRPs. Specifically, the present inventors found that it is possible to accurately measure the CRP concentration in a high CRP concentration range by using recombinant CRPs, 55% or more of which have a pyroglutamylated N-terminal, as measured by intact MS. Thus, the present invention has been completed.

Specific aspects of the present invention are as shown below.

Item 1. Recombinant C-reactive proteins produced by genetic recombination, 55% or more of the C-reactive proteins having a pyroglutamylated N-terminal.

Item 2. The recombinant C-reactive proteins according to Item 1, wherein 65% or more of the C-reactive proteins have a pyroglutamylated N-terminal.

Item 3. The recombinant C-reactive proteins according to Item 1, wherein 75% or more of the C-reactive proteins have a pyroglutamylated N-terminal.

Item 4. The recombinant C-reactive proteins according to Item 1, wherein 85% or more of the C-reactive proteins have a pyroglutamylated N-terminal.

Item 5. The recombinant C-reactive proteins according to any one of Items 1 to 4, wherein the recombinant C-reactive proteins are bacterial recombinant proteins.

Item 6. The recombinant C-reactive proteins according to Item 5, wherein the bacterium is Escherichia coli.

Item 7. The recombinant C-reactive proteins according to any one of Items 1 to 6, wherein the C-reactive proteins are derived from a human.

Item 8. The recombinant C-reactive proteins according to any one of Items 1 to 7, wherein the C-reactive proteins comprise any of the following polypeptides (a) to (c):

(a) a polypeptide represented by SEQ ID No: 1 or SEQ ID No: 2;

(b) a polypeptide comprising an amino acid sequence including substitution, deletion, insertion, and/or addition of one or more amino acid residues in the amino acid sequence represented by SEQ ID No: 1 or SEQ ID No: 2, and having antigenicity against anti-C-reactive protein antibody; and

(c) a polypeptide comprising an amino acid sequence having 90% or more identity to the amino acid sequence represented by SEQ ID No: 1 or SEQ ID No: 2, and having antigenicity against anti-C-reactive protein antibody.

Item 9. A calibrator comprising the recombinant C-reactive proteins according to any one of Items 1 to 8.

Item 10. Control serum comprising the recombinant C-reactive proteins according to any one of Items 1 to 8.

Item 11. A method for quantifying C-reactive proteins in a sample using the calibrator comprising the recombinant C-reactive proteins according to Item 9.

Item 12. A method for quantifying C-reactive proteins in a sample using the control serum comprising the recombinant C-reactive proteins according to Item 10.

Item 13. The method for quantifying C-reactive proteins in a sample according to Item 11 or 12, by latex turbidimetric immunoassay using latex particles on which anti-C-reactive protein antibody is immobilized.

### Advantageous Effects of Invention

The recombinant CRPs of the present invention can improve the accuracy of measurements by latex turbidimetric immunoassay in a high CRP concentration range, and is useful as diagnostic raw materials for use in control serum or calibrators.

### BRIEF DESCRIPTION OF DRAWINGS

FIG. 1 shows the results of performing SDS-PAGE of recombinant CRP1 in Example 3.

FIG. 2 shows the results of performing SDS-PAGE of recombinant CRP2 in Example 3.

FIG. 3 shows the results of performing comparative study of expanded spectra of average 10-valent ions with an elution time of 1.7 to 2.0 minutes in LC/MS of various types of CRP1 in Example 5.

FIG. 4 shows the results of performing comparative study of expanded spectra of average 10-valent ions with an elution time of 1.7 to 2.0 minutes in LC/MS of various types of CRP2 in Example 5.

### DESCRIPTION OF EMBODIMENTS

### Polypeptide of C-Reactive Proteins

Examples of the recombinant CRPs of the present invention include CRPs derived from mammals, such as humans, dogs, cats, mice, rats, rabbits, or goats. Preferred among these are CRPs derived from humans, dogs, or cats; and particularly preferred are CRPs derived from humans.

An embodiment of the present invention is CRPs comprising any of the following polypeptides (a) to (c):

(a) a polypeptide represented by SEQ ID No: 1 or SEQ ID No: 2;

(b) a polypeptide comprising an amino acid sequence including substitution, deletion, insertion, and/or addition of one or more amino acid residues in the amino acid sequence represented by SEQ ID No: 1 or SEQ ID No: 2, and having antigenicity against anti-C-reactive protein antibody; and

(c) a polypeptide comprising an amino acid sequence having 90% or more identity to the amino acid sequence represented by SEQ ID No: 1 or SEQ ID No: 2, and having antigenicity against anti-C-reactive protein antibody.

In the polypeptide (a), SEQ ID No: 1 or SEQ ID No: 2 is an amino acid sequence of mature CRP consisting of 206 amino acids. When the recombinant CRPs of the present invention are expressed outside bacteria, such as Gram-negative bacteria, an appropriate secretion signal suitable for the host may be added. The amino acid sequence in that case has a total length of 227 amino acids, as shown in SEQ ID No: 3 or SEQ ID No: 4. Among these, 206 amino acids from positions 22 to 227 correspond to the mature CRP of SEQ ID No: 1 or SEQ ID No: 2, and the sequence from methionine to position 21 is the amino acid sequence of the secretion signal. When the recombinant CRPs of the present invention are expressed inside bacteria, the secretion signal may be deleted.

The recombinant CRPs of the present invention are not limited to (a) above, and may be those comprising:

(b) a polypeptide comprising an amino acid sequence including substitution, deletion, insertion, and/or addition of one or more amino acid residues in the amino acid sequence represented by SEQ ID No: 1 or SEQ ID No: 2, and having antigenicity against anti-C-reactive protein antibody; or

(c) a polypeptide comprising an amino acid sequence having 90% or more identity to the amino acid sequence represented by SEQ ID No: 1 or SEQ ID No: 2, and having antigenicity against anti-C-reactive protein antibody.

In the polypeptide (b), the lower limit of the number of “more amino acid residues” is 2. The upper limit is not particularly limited as long as the antigenicity against anti-C-reactive protein antibody can be maintained; however, it is necessary to be within a range in which the three-dimensional structure of the protein of amino acid residues and the antigenicity against anti-C-reactive protein antibody are not significantly impaired. For example, the upper limit is a number corresponding to less than 20% of all amino acids, preferably less than 15%, more preferably less than 10%, even more preferably less than 5%, and still even more preferably less than 1%. In other words, the number of amino acid residues is, for example, 41 or less, preferably 31 or less, more preferably 21 or less, even more preferably 10 or less, still even more preferably 5 or less, still even more preferably 4 or less, and further still even more preferably 3 or less.

In the polypeptide (c), the identity to the amino acid sequence shown in (a) is preferably 80% or more. The identity is preferably 85% or more, more preferably 90% or more, even more preferably 95% or more, still even more preferably 98% or more, and further still even more preferably 99% or more.

The amino acid sequence identity can be calculated using analysis tools available commercially or via telecommunication lines (the internet). In the present invention, the identity is calculated by selecting blastp on the BLAST website, which is a homology search program published by the National Center for Biotechnology Information (NCBI), and using the default parameters.

Whether a polypeptide has antigenicity against anti-C-reactive protein antibody is determined based on whether latex particles undergo aggregation, and the CRP concentration can be measured in the “Method for Measuring Concentration of C-Reactive Proteins” described later.

The variant of the protein having antigenicity against anti-C-reactive protein antibody and its gene can be obtained, for example, by modifying the base sequence encoding the amino acid represented by SEQ ID No: 1 or SEQ ID No: 2 using a PCR method and a commercially available kit, such as Transformer Mutagenesis Kit produced by Clontech, EXOIII/Mung Bean Deletion Kit produced by Stratagene, QuickChang Site-Directed Mutagenesis Kit produced by Stratagene, or KOD-Plus-Mutagenesis Kit produced by Toyobo Co., Ltd. The antigenicity of the protein encoded by the obtained gene can be confirmed, for example, by introducing the obtained gene into Escherichia coli to prepare a transformant, culturing the transformant to generate protein, and measuring the transformant, a disrupted cell suspension of the transformant, or purified protein by the “Method for Measuring Concentration of C-Reactive Proteins” described later.

### DNA of C-Reactive Proteins

Examples of the DNA encoding the recombinant CRPs of the present invention include DNAs encoding CRPs derived from mammals, such as humans, dogs, cats, mice, rats, rabbits, or goats. Preferred among these are DNAs encoding CRPs derived from humans, dogs, or cats; and particularly preferred are DNAs encoding CRPs derived from humans.

An embodiment of the present invention is CRPs comprising any of the following DNAs (d) to (f):

(d) DNA encoding the amino acid sequence of the CRPs of any of (a) to (c);

(e) DNA comprising a base sequence represented by SEQ ID No: 5 or SEQ ID No: 6;

(f) DNA encoding a polypeptide comprising a base sequence including substitution, deletion, insertion, and/or addition of one or more bases in the base sequence represented by SEQ ID No: 5 or SEQ ID No: 6, and having antigenicity against anti-C-reactive protein antibody; and

(g) DNA encoding a polypeptide comprising a base sequence having 80% or more identity to the base sequence represented by SEQ ID No: 5 or SEQ ID No: 6, and having antigenicity against anti-C-reactive protein antibody.

In the DNA (d), the amino acid sequence of the CRPs of the present invention is the amino acid sequence of the CRPs shown in any of (a) to (c). The options for the DNA of the present invention are not particularly limited when there are multiple codons corresponding to the amino acids in the amino acid sequence. Specific examples include (e) DNA comprising the base sequence represented by SEQ ID No: 5 or SEQ ID No: 6.

When a secretion signal sequence is added as described above, it is preferable to use DNA represented by SEQ ID No: 7 or SEQ ID No: 8. SEQ ID No: 7 or SEQ ID No: 8 encodes the mature CRP with 206 amino acids from positions 22 to 227 in the total length of CRP represented by the amino acid sequence of SEQ ID No: 3 or SEQ ID No: 4, and a part corresponding to the secretion signal from methionine to position 21.

The DNA of the present invention is not limited to those mentioned above, and may be:

(f) DNA encoding a polypeptide comprising a base sequence including substitution, deletion, insertion, and/or addition of one or more bases in the base sequence represented by SEQ ID No: 5 or SEQ ID No: 6, and having antigenicity against anti-C-reactive protein antibody; or

(g) DNA encoding a polypeptide comprising a base sequence having 80% or more identity to the base sequence represented by SEQ ID No: 5 or SEQ ID No: 6, and having antigenicity against anti-C-reactive protein antibody.

When the DNA encoding the CRPs of the present invention is incorporated into a host other than organisms derived from Escherichia coli or the like to express the CRPs of the present invention, the base sequence may be changed according to the codon usage of the host organism, in order to improve the expression efficiency.

In the DNA (f), the lower limit of the number of “more bases” is 2. The upper limit is not particularly as long as the antigenicity against anti-C-reactive protein antibody of the polypeptide encoded by the DNA can be maintained; however, it is necessary to be within a range in which the three-dimensional structure of the protein of amino acid residues and the antigenicity against anti-C-reactive protein antibody are not significantly impaired. For example, the upper limit is a number corresponding to less than 20% of all amino acids of the polypeptide before modification, preferably less than 15%, more preferably less than 10%, even more preferably less than 5%, and still even more preferably less than 1%. In other words, the number of bases is, for example, 124 or less (the number of bases corresponding to 20% of all amino acids), preferably 93 or less (15%), more preferably 62 or less (10%), even more preferably 31 or less (5%), still even more preferably 20 or less, still even more preferably 15 or less, still even more preferably 10 or less, still even more preferably 5 or less, still even more preferably 4 or less, and further still even more preferably 3 or less.

In the DNA (g), the identity to the base sequence represented by SEQ ID No: 5 or SEQ ID No: 6 is preferably 80% or more, more preferably 85% or more, even more preferably 90% or more, still even more preferably 95% or more, still even more preferably 98% or more, and further still even more preferably 99% or more.

The base sequence identity can be calculated using analysis tools available commercially or via telecommunication lines (the internet). In the present invention, the identity is calculated by selecting blastn on the BLAST website, which is a homology search program published by the National Center for Biotechnology Information (NCBI), and using the default parameters.

Whether a DNA encodes a polypeptide having antigenicity against anti-C-reactive protein antibody can be determined in such a manner that the DNA is incorporated into a commercial expression vector and expressed in a suitable host, and the antigenicity against anti-C-reactive protein antibody of the obtained polypeptide can be determined based on whether latex particles undergo aggregation, and the CRP concentration can be measured in the “Method for Measuring Concentration of C-Reactive Proteins” described later.

### Method for Producing C-Reactive Proteins

Another embodiment of the present invention is a vector into which the above DNA is incorporated, a transformant comprising the vector, or a method for producing recombinant CRPs, comprising culturing the transformant. The recombinant CRPs of the present invention can be easily prepared by inserting the gene into a suitable vector to prepare a recombinant vector, and transforming a suitable host cell with the recombinant vector to prepare a transformant, and culturing the transformant.

The vector is not particularly limited as long as it can replicate and retain or autonomously proliferate in various host cells of prokaryotic and/or eukaryotic cells. Examples include plasmid vectors, phage vectors, viral vectors, and the like. The preparation of the recombinant vector is not particularly limited, and may be performed according to a general method. For example, the preparation can easily be performed by linking the gene of the CRPs of the present invention to such a vector using an appropriate restriction enzyme and ligase or, if necessary, further a linker or adaptor DNA. A gene fragment amplified using a DNA polymerase that adds a single base to the amplified end, such as Taq DNA polymerase, can also be connected to the vector by TA cloning.

Moreover, the host cell is not particularly limited as long as a recombinant expression system is established. Preferred examples include microorganisms, such as Escherichia coli, Bacillus subtilis, actinomycetes, Aspergillus oryzae, and yeast, as well as insect cells, animal cells, higher plants, and the like. More preferred are microorganisms, and particularly preferred is Escherichia coli (e.g., K12 strain and B strain). The preparation of the transformant is not particularly limited, and may be performed according to a general method. When the host is Escherichia coli, Escherichia coli C600, Escherichia coli HB101, Escherichia coli DH5a, Escherichia coli JM109, Escherichia coli BL21, or the like is used. Examples of vectors include pBR322, pUC19, pBluescript, pQE, pET, and the like. When the host is yeast, preferred examples include Saccharomyces cerevisiae, Schizosaccharomyces pombe, Candida utilis, Pichia pastoris, and the like. Examples of vectors include pAUR101, pAUR224, pYE32, and the like. When the host is a filamentous fungus, examples include Aspergillus oryzae, Aspergillus niger, and the like.

When the obtained transformant is cultured for a certain period of time under culture conditions suitable for the host cell, the recombinant CRPs of the present invention are expressed by the incorporated gene and accumulated in the transformant.

The recombinant CRPs of the present invention accumulated in the transformant can be used in an unpurified form; however, it is preferable to use a purified form. The purification method is not particularly limited, and can be performed, for example, by homogenizing the transformant after culture or a cultured product thereof in a suitable buffer, obtaining a cell extract by sonication, surfactant treatment, or the like, and then appropriately combining separation techniques commonly used for protein separation and purification. Examples of such separation techniques include, but are not limited to, methods using the difference in solubility, such as salting out and solvent precipitation; methods using the difference in molecular weight, such as dialysis, ultrafiltration, gel filtration, unmodified polyacrylamide gel electrophoresis (PAGE), and sodium dodecyl sulfate-polyacrylamide gel electrophoresis (SDS-PAGE); methods using charges, such as ion exchange chromatography and hydroxyapatite chromatography; methods using specific affinity, such as affinity chromatography using a phosphorylcholine-immobilized column; methods using the difference in hydrophobicity, such as reverse phase high-performance liquid chromatography; methods using the difference in isoelectric point, such as isoelectric focusing electrophoresis; and the like. For example, purified preparations can be obtained by separation by gel filtration using Sephadex gel (produced by GE Healthcare Bio-Sciences Corp) or the like, or column chromatography using DEAE Sepharose CL-6B (produced by GE Healthcare Bio-Sciences Corp), Octyl-Sepharose CL-6B (produced by GE Healthcare Bio-Sciences Corp), or the like, and purification.

### Method for Cyclizing N-Terminal of C-Reactive Proteins

55% or more of all of the recombinant CRPs of the present invention are recombinant CRPs with a pyroglutamylated N-terminal, and specifically recombinant CRPs with an N-terminal cyclization rate calculated as 55% or more.

In the present invention, the “N-terminal cyclization rate” is an indicator showing the ratio of recombinant CRPs with a pyroglutamylated N-terminal among recombinant CRPs, and is calculated according to the “Method for Measuring N-Terminal Cyclization Rate of C-Reactive Proteins” described later. When the N-terminal cyclization rate is calculated by the “Method for Measuring N-Terminal Cyclization Rate of C-Reactive Proteins” described later, it is preferable to hold 55% or more of recombinant CRPs, preferably 60% or more, more preferably 65% or more, even more preferably 70% or more, still even more preferably 75% or more, still even more preferably 80% or more, still even more preferably 85% or more, still even more preferably 90% or more, still even more preferably 95% or more, and further still even more preferably 98% or more.

In the present invention, the “N-terminal” refers to the N-terminal of mature CRP from which a secretion signal sequence is removed, and specifically refers to glutamine at position 1 of the amino acid sequence represented by SEQ ID No: 1 or SEQ ID No: 2 (or at position 22 of SEQ ID No: 3 or SEQ ID No: 4), or pyroglutamyl acid after the glutamine at position 1 undergoes an intramolecular condensation reaction. The glutamine at position 1 is converted into pyroglutamic acid by cyclization treatment.

In the present invention, the “cyclization treatment” refers to a treatment that promotes the conversion of CRP from a state where the above N-terminal is glutamine (hereinafter referred to as “uncyclized”) to a state where the above N-terminal is pyroglutamyl acid (hereinafter referred to as “cyclized”). An enzymatic or non-enzymatic method may be selected. The enzymatic method can be performed, for example, by reacting enzyme, such as glutaminyl cyclase, at an appropriate temperature; however, the enzyme and temperature condition are not limited thereto. For the non-enzymatic method, it is necessary to set the type of buffer, pH, treatment temperature, treatment time, CRP concentration, etc.; however, any conditions that do not adversely affect CRP, uncyclized CRP, and cyclized CRP may be used, and these are not particularly limited. An example is shown below.

Examples of the buffer used in the cyclization treatment include Good's buffers, such as acetic acid buffer, MES buffer, and PIPES buffer; phosphoric acid buffer, Tris-HCl buffer, boric acid buffer, glycine buffer, and the like. The pH condition is preferably in the range of pH 7 to pH 12, and more preferably pH 7 to pH 10. The temperature condition is preferably 4° C. to 55° C., and more preferably 37° C. to 50° C. The CRP concentration is preferably 0.1 mg/dL to 500 mg/dL, and more preferably 10 mg/dL to 300 mg/dL. The reaction time is preferably 30 minutes to 4 weeks, and more preferably 16 hours to 3 weeks.

### Method for Measuring Concentration of C-Reactive Proteins

In the present invention, the CRP concentration is measured under the following conditions. In the CRP concentration measurement method, latex particles with immobilized anti-C-reactive protein antibody and CRP (test substance) in a test sample cause an antigen-antibody reaction, and the CRP concentration is measured from the degree of aggregation of the latex particles. In the present invention, the “CRP concentration” refers to a value measured by the following method, unless otherwise specified.

### Reagents

CRP latex X2 “Seiken” R1 reagent (buffer), produced by Denka Seiken Co., Ltd.

CRP latex X2 “Seiken” R2 reagent (latex (anti-human CRP polyclonal antibody (rabbit)-bound latex) suspension), produced by Denka Seiken Co., Ltd.

CRPX2 Standard Liquid H, produced by Denka Seiken Co., Ltd.

### Measurement Sample

The measurement sample is a CRP solution, and is diluted with 20 mM Tris-HCl buffer (0.14 M sodium chloride, 2 mM calcium chloride; pH 7.5), as needed, before use. When a sample with a specific CRP concentration is prepared, the dilution factor is determined based on the protein concentration value-assigned in the Bradford protein assay.

### Measurement Method

Using the above measurement sample, and the above R1 reagent and R2 reagent, the CRP concentration (mg/dL) of the sample is measured under the following conditions using Hitachi 7180 Fully Automatic Biochemistry Analyzer. The CRP concentration is calculated by formula (I).

### Sample: 2.2 μL

R1 reagent: 120 μL
R2 reagent: 120 μL
Measurement method: two-point end method (18-34)
Dominant wavelength: 546 nm
Complementary wavelength: 800 nm

CRP concentration (mg/dL)={measured value (TEST)−measured value (BLANK)}×dilution factor of CRP solution (I)

In the present invention, as the method for determining whether the reactivity of the recombinant CRPs to the latex reagents in a high CRP concentration range is improved, when the deviation between the CRP concentration value of natural human CRP in the high CRP concentration range measured by the above method, and the CRP concentration value of each recombinant CRP in the high CRP concentration range is 5% or less, it is determined that the reactivity is improved. The high CRP concentration range in the present invention refers to the CRP concentration range of 10 to 30 mg/dL, but is not particularly limited thereto.

### Method for Measuring N-Terminal Cyclization Rate of C-Reactive Proteins

In the present invention, the N-terminal cyclization rate of the recombinant CRPs is measured under the following conditions. In this measurement method, the 10-valent ion intensities of the spectra of uncyclized CRP and cyclized CRP are measured by mass spectrometry, and the ratio of uncyclized CRP and cyclized CRP is calculated from each ion intensity. In the present invention, the “spectrum of uncyclized CRP” refers to a spectrum corresponding to a molecular weight of 23045, and the “spectrum of cyclized CRP” refers to a spectrum corresponding to a molecular weight of 23027. In the present invention, the “N-terminal cyclization rate of CRP” specifically refers to a value measured by the following method, unless otherwise specified.

### Measurement Sample

The measurement sample is a CRP solution, and is diluted with ultrapure water, as needed, before use.

### Measurement Method

The spectra of uncyclized CRP and cyclized CRP in the above measurement sample are measured under the following conditions using LC/MS devices.

LC conditions
Device: ACQUITY UPLC, produced by Waters
Column: MassPREP Micro Desalting Column (20 μm, 2.1×5 mm),
produced by Waters
Mobile phase:

A: water/formic acid mixture (1000:1),

B: IPA/ACN/methanol/formic acid mixture (500:300:200:1)

Column temperature: 50° C.
Injection amount: 5 μL
MS conditions
Device: micrOTOF, produced by Bruker Daltonics
Ionization method: ESI Positive

### Method for Calculating N-Terminal Cyclization Rate

The 10-valent ion intensity of the average spectrum with an elution time of 1.7 to 2.0 minutes for each CRP spectrum obtained by the above measurement method is used to calculate the ratio of uncyclized CRP and cyclized CRP. The “ratio” in the present invention refers to the ratio of the ion intensity of cyclized CRP when the sum of the two ion intensities of uncyclized CRP and cyclized CRP is 100, and is calculated by the following formula (II).

N-terminal cyclization rate (%)=ion intensity of cyclized CRP/(ion intensity of uncyclized CRP+ion intensity of cyclized CRP)×100 (II)

The present invention is described in more detail below with reference to Examples. The present invention is not particularly limited by the Examples.

### Example 1: Introduction of Variation and Acquisition of Transformant

### (1) Introduction of Variation

An artificial synthetic gene of SEQ ID No: 7 or SEQ ID No: 8 obtained by linking Escherichia coli-derived alkaline phosphatase secretion signal sequence

(ATGAAACAAAGCACTATTGCACTGGCACTCTTACCGTTACTGTTTAC
CCCTGTGACAAAAGCC)

and human-derived mature CRP sequence of SEQ ID No: 5 or SEQ ID No: 6 was used as a template, and primers of SEQ ID Nos: 9 and 10 were used to amplify a CRP gene. SEQ ID No: 9 is a forward primer, and SEQ ID No: 10 is a reverse primer. Restriction enzyme site NdeI or restriction enzyme site BamHI is added to the primers. The amplified gene fragment, vector plasmid pBluescript KSN(+) cleaved with the restriction enzymes NdeI and BamHI, and In-Fusion Reaction Mix (produced by Takara Bio Inc.) were added, followed by incubation, thereby constructing a plasmid. In this manner, recombinant plasmid pBKSN_CRP1 including SEQ ID No: 7 and recombinant plasmid pBKSN_CRP2 including SEQ ID No: 8, which were designed to express a large amount of CRP gene, were obtained.

### (2) Acquisition of Transformant

Using the plasmid constructed in (1), Escherichia coli JM109 strain competent cells (produced by Toyobo Co., Ltd.) were transformed, cultured in SOC medium for 1 hour at 37° C., and then developed on LB agar medium (containing 1.0% glucose and 50 μg/mL ampicillin) to obtain a colony as a transformant. The transformant obtained by introducing pBKSN_CRP1 was named Escherichia coli JM109 (pBKSN_CRP1). Further, a transformant obtained by introducing pBKSN_CRP2 in the same manner as above was named Escherichia coli JM109 (pBKSN_CRP2).

### Example 2: Expression of CRP Gene in Escherichia coli

The colony of the transformant Escherichia coli JM109 (pBKSN_CRP1) obtained in Example 1 was inoculated in 5 mL of LB liquid medium (containing 1.0% glucose and 100 μg/mL ampicillin) sterilized in vitro, and cultured at 37° C. for 16 hours. The obtained culture broth was used as a seed culture broth and inoculated in 500 mL of LB liquid medium (containing 0.5% glycerol, 0.05% calcium chloride, 1 mM IPTG, and 50 μg/mL ampicillin) in 10 2-L Sakaguchi flasks. Then, the cells were cultured at a shaking speed of 180 rpm at 30° C. for 24 hours. At the end of the culture, the bacterial bodies were collected by centrifugation, suspended in 20 mM Tris-HCl buffer (0.14 M sodium chloride, 2 mM calcium chloride, pH 7.5), then crushed in a French press (produced by Niro Soavi), and further centrifuged to thereby obtain the supernatant liquid as a crude purified liquid 1. In addition, a crude purified liquid 2 was obtained from Escherichia coli JM109 (pBKS_N CRP2) in the same manner as above.

### Example 3: Purification of Recombinant CRPs

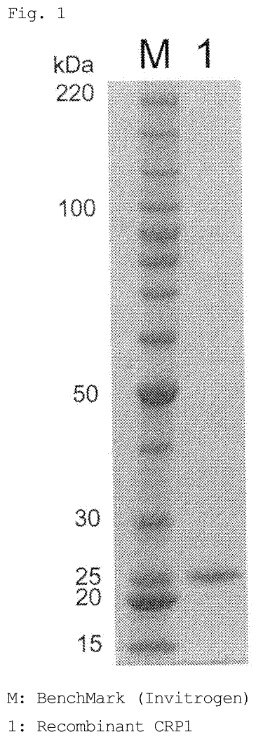

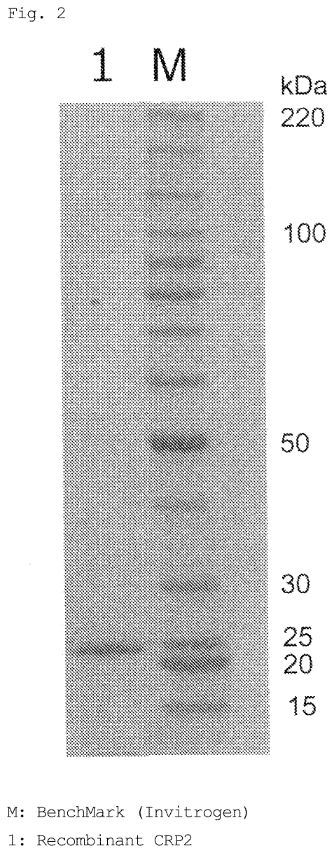

The crude purified liquid 1 obtained in Example 2 was subjected to affinity purification using Pierce™ p-Aminophenyl Phosphoryl Choline Agarose (produced by Thermo Scientific). The resin equilibrated with the buffer used in Example 2, i.e., 20 mM Tris-HCl buffer (0.14 M sodium chloride, 2 mM calcium chloride, pH 7.5), was mixed and absorbed with the crude purified liquid. The resulting resin was washed with the above buffer, and eluted in 20 mM Tris-HCl buffer (0.14 M sodium chloride, 2 mM EDTA, pH 7.5) to obtain a recombinant CRP solution 1. The solution was further replaced with the buffer used in Example 2, i.e., 20 mM Tris-HCl buffer (0.14 M sodium chloride, 2 mM calcium chloride, pH 7.5), while removing EDTA by water concentration using a hollow fiber membrane, and further concentrated to a suitable concentration with a centrifugal ultrafiltration filter (produced by Merck), thereby obtaining high-purity recombinant CRP1. FIG. 1 shows the results of performing SDS-PAGE using the obtained recombinant CRP1. As a result, improving the purity to a level at which impure protein could not be detected by SDS-PAGE succeeded. In addition, high-purity recombinant CRP2 was also obtained from the crude purified liquid 2 in the same manner as above. FIG. 2 shows the results of performing SDS-PAGE on the obtained CRP2. As in the case of CRP1, improving the purity to a level at which impure protein could not be detected by SDS-PAGE succeeded.

### Example 4: N-Terminal Cyclization Treatment of Recombinant CRPs

The recombinant CRP1 obtained in Example 3 was heated at 37° C. for 8 days to obtain cyclized recombinant CRP1 in which the N-terminal of recombinant CRP was cyclized by pyroglutamylation. In the cyclization treatment, the buffer used in Example 2, i.e., 20 mM Tris-HCl buffer (0.14 M sodium chloride, 2 mM calcium chloride, pH 7.5), was used, and the CRP concentration was 300 mg/dL. Further, cyclized recombinant CRP2 was obtained from the recombinant CRP2 in the same manner as above.

### Example 5: Measurement of N-Terminal Cyclization Rate

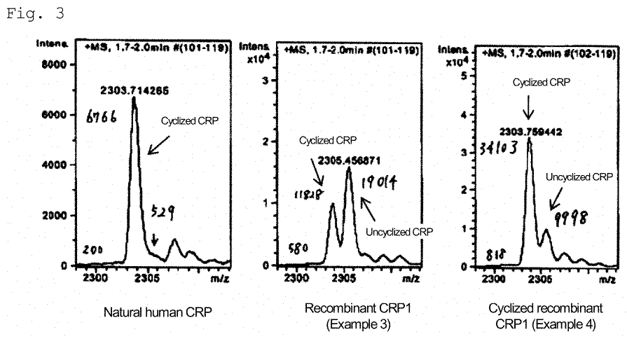

Using the recombinant CRP1 and recombinant CRP2 obtained in Example 3, the cyclized recombinant CRP1 and cyclized recombinant CRP2 obtained in Example 4, and natural human CRP (produced by Yashraj) as a positive control, CRP solutions were prepared using ultrapure water to a CRP concentration of 150 mg/dL, followed by measurement under the LC/MS conditions as described above (see Table 1). FIG. 3 shows an enlargement of the average 10-valent ion with an elution time of 1.7 to 2.0 minutes in the mass spectrum of each sample of recombinant CRP1.

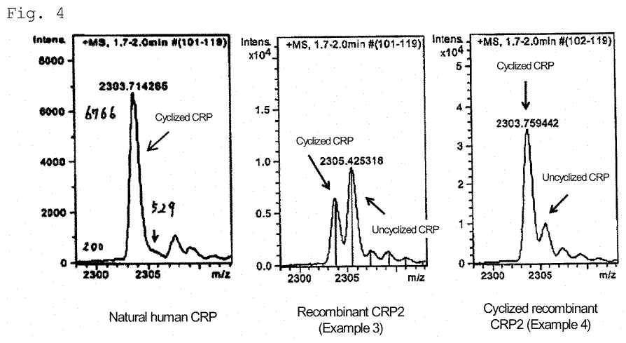

m/z 2305.45 represents the spectrum of uncyclized CRP, and m/z 2303.74 represents the spectrum of cyclized CRP. In the natural human CRP, the spectrum of cyclized CRP was shown prominently. On the other hand, in the recombinant CRP1 obtained in Example 3, it was shown that the spectrum of uncyclized CRP was higher than the spectrum of cyclized CRP. In contrast, in the cyclized recombinant CRP1 obtained in Example 4, it was shown that the spectrum of cyclized CRP was clearly higher than the spectrum of uncyclized CRP. It was shown that the N-terminal cyclization treatment of Example 4 promoted the pyroglutamylation of CRP. Similar results were obtained for the recombinant CRP2 and the cyclized recombinant CRP2, as shown in FIG. 4.

TABLE 1

| | Flow | Mobile |
|---|---|---|
| Time | rate | phase |
| (min) | (mL/min) | B (%) |
| 0.01 | 0.40 | 5 |
| 0.50 | 0.40 | 5 |
| 0.51 | 0.16 | 5 |
| 2.00 | 0.16 | 95 |
| 2.10 | 0.40 | 5 |
| 2.70 | 0.40 | 95 |
| 2.80 | 0.40 | 5 |
| 3.40 | 0.40 | 95 |
| 3.50 | 0.40 | 5 |
| 4.00 | 0.40 | 5 |

In the recombinant CRP1 and recombinant CRP2 obtained in Example 3, the cyclized recombinant CRP1 and cyclized recombinant CRP2 obtained in Example 4, and natural human CRP, the N-terminal cyclization rate of each CRP was calculated by the above formula (II) from the ratio of uncyclized CRP and cyclized CRP from each ion intensity based on the 10-valent ion intensities of the spectra of uncyclized CRP and cyclized CRP measured under the above LC/MS conditions. Table 2 shows the N-terminal cyclization rates of the recombinant CRP1, cyclized recombinant CRP1, and natural human CRP. For the cyclized recombinant CRP1 obtained in Example 4, the N-terminal cyclization rate of the cyclized recombinant CRP1 obtained on days 1, 3, 6, and 8 of heating was calculated by formula (II). Further, Table 3 shows the N-terminal cyclization rates of the recombinant CRP2, cyclized recombinant CRP2, and natural human CRP. For the cyclized recombinant CRP2 obtained in Example 4, the N-terminal cyclization rate of the cyclized recombinant CRP2 obtained on days 1, 3, 6, and 8 of heating was calculated by formula (II).

The N-terminal cyclization rate of the natural human CRP is 95%, indicating that 95% of the total is cyclized CRP. On the other hand, the N-terminal cyclization rates of the recombinant CRP1 and recombinant CRP2 obtained in Example 3 are 40%, indicating that cyclized CRP is present in less than half of the total. In contrast, the cyclized recombinant CRP1 and cyclized recombinant CRP2 obtained in Example 4 showed an N-terminal cyclization rate of 42% on day 1 of heating, an N-terminal cyclization rate 54% on day 3 of heating, an N-terminal cyclization rate of 67% on day 6 of heating, and an N-terminal cyclization rate of 78% on day 8 of heating, indicating that the proportion of cyclized recombinant CRP tended to increase as the heating time increased.

TABLE 2

| | N-terminal |
|---|---|
| | cyclization |
| Sample | rate (%) |
| Example 3: recombinant CRP1 | 40 |
| Example 4: cyclized recombinant | 42 |
| CRP1, day 1 of heating | |
| Example 4: cyclized recombinant | 54 |
| CRP1, day 3 of heating | |
| Example 4: cyclized recombinant | 67 |
| CRP1, day 6 of heating | |
| Example 4: cyclized recombinant | 78 |
| CRP1, day 8 of heating | |
| Natural human CRP | 95 |

TABLE 3

| | N-terminal |
|---|---|
| | cyclization |
| Sample | rate (%) |
| Example 3: recombinant CRP2 | 40 |
| Example 4: cyclized recombinant | 42 |
| CRP2, day 1 of heating | |
| Example 4: cyclized recombinant | 54 |
| CRP2, day 3 of heating | |
| Example 4: cyclized recombinant | 67 |
| CRP2, day 6 of heating | |
| Example 4: cyclized recombinant | 78 |
| CRP2, day 8 of heating | |
| Natural human CRP | 95 |

### Example 6: Measurement of CRP Concentration by Latex Reagent

Bovine serum albumin (produced by Sigma) was diluted with ultrapure water to 0.1, 0.2, 0.4, and 0.75 mg/mL to prepare standard solutions. 600 μL of protein assay concentrated dye reagent (produced by Bio-Rad) and 2.4 mL of ultrapure water were added to 60 μL of each standard solution, and the mixture was allowed to stand at room temperature for 5 minutes. Then, the absorbance at 595 nm was measured. A calibration curve was prepared from the measured absorbance at 595 nm and the bovine serum albumin concentration. The recombinant CRP1 and recombinant CRP2 obtained in Example 3, the cyclized recombinant CRP1 and cyclized recombinant CRP2 obtained in Example 4, and natural human CRP were diluted with ultrapure water to a suitable concentration using the same reagent under the same conditions as those for the above standard solutions, and the absorbance at 595 nm was measured. From the measured values of these CRPs, the protein concentration was calculated from the above calibration curve. Based on the above protein concentration, CRP solutions were prepared by dilution with 20 mM Tris-HCl buffer (0.14 M sodium chloride, 2 mM calcium chloride, pH 7.5) to protein concentrations of 1 mg/dL, 5 mg/dL, 10 mg/dL, and 30 mg/dL.

For the CRP concentration of each of the above CRP solutions, a first reagent (a buffer of CRP-latex X2 produced by Denka Seiken Co., Ltd.) and a second reagent (an anti-human CRP polyclonal antibody (rabbit)-bound latex suspension) were combined, and the CRP concentration-dependent formation of particle aggregates was measured using Hitachi 7180 Fully Automatic Biochemistry Analyzer. Specifically, 120 μL of the first reagent was added to 2.2 μL of each of the CRP solutions, the mixture was heated at 37° C. For 5 minutes, and then 120 μL of the second reagent was added and stirred. Thereafter, the change in absorbance (ΔmAbs) associated with the aggregate formation for 5 minutes was measured at a dominant wavelength of 546 nm and a complementary wavelength of 800 nm.

Table 4 shows the measured values of the recombinant CRP1 obtained in Example 3 and the cyclized recombinant CRP1 obtained in Example 4, and the relative values at each concentration with respect to the measured value of the natural human CRP. Table 4 also shows the N-terminal cyclization rates of the various CRPs calculated in Example 5. Furthermore, Table 5 shows the measured values of the recombinant CRP1 obtained in Example 3, the cyclized recombinant CRP1 obtained in Example 4, and the natural human CRP. As in the above case, Table 6 shows the measured values of the recombinant CRP2 obtained in Example 3 and the cyclized recombinant CRP2 obtained in Example 4, and the relative values at each concentration with respect to the measured value of the natural human CRP. Table 6 also shows the N-terminal cyclization rates of the various CRPs calculated in Example 5. Furthermore, Table 7 shows the measured values of the recombinant CRP2 obtained in Example 3, the cyclized recombinant CRP2 obtained in Example 4, and the natural human CRP.

Tables 4 and 6 confirmed that in the recombinant CRP1 and recombinant CRP2 with an N-terminal cyclization rate of 40% or 42%, the relative values with respect to the measured value of the natural human CRP at the points of 10 mg/dL and 30 mg/dL were 89 to 94%, and that there was a deviation of 6 to 11% in the measured values. On the other hand, in the cyclized recombinant CRP1 and cyclized recombinant CRP2 with an N-terminal cyclization rate of 54%, 67%, or 78%, the relative values with respect to the measured value of the natural human CRP at the points of 10 mg/dL and 30 mg/dL were 95 to 104%, and the deviation from the measured values was suppressed within 5%. These results indicated that in the cyclized recombinant CRP1 and cyclized recombinant CRP2 with an N-terminal cyclization rate of 55% or more, the deviation from the natural human CRP could be suppressed within 5% even in the high CRP concentration ranges of 10 mg/dL and 30 mg/dL.

TABLE 4

| | | Example 4 | Example 4 | Example 4 | Example 4 |
|---|---|---|---|---|---|
| | | cyclized | cyclized | cyclized | cyclized |
| | Example 3 | CRP1 | CRP1 | CRP1 | CRP1 |
| | recombinant | day 1 of | day 3 of | day 6 of | day 8 of |
| | CRP1 | heating | heating | heating | heating |
| N-terminal | 40% | 42% | 54% | 67% | 78% |
| cyclization | | | | | |
| rate | | | | | |
| Theoretical | | | | | |
| value | | | | | |
| (mg/dL) | Relative value (recombinant CRP/natural human ORP) | | | | |
| 1 | 103% | 100% | 102% | 104% | 102% |
| 5 | 98% | 96% | 100% | 104% | 101% |
| 10 | 94% | 89% | 97% | 102% | 99% |
| 30 | 91% | 91% | 95% | 100% | 101% |

TABLE 5

| | | Example 4 | Example 4 | Example 4 | Example 4 | |
|---|---|---|---|---|---|---|
| | | cyclized | cyclized | cyclized | cyclized | |
| | Example 3 | CRP1 | CRP1 | CRP1 | CRP1 | Natural |
| | recombinant | day 1 of | day 3 of | day 6 of | day 8 of | human |
| | CRP1 | heating | heating | heating | heating | CRP |
| N-terminal | 40% | 42% | 54% | 67% | 78% | 95% |
| cyclization rate | | | | | | |
| Theoretical | Measured value (mg/dL) | | | | | |
| value (mg/dL) | | | | | | |
| 1 | 0.79 | 0.76 | 0.77 | 0.79 | 0.78 | 0.76 |
| 5 | 4.80 | 4.69 | 4.88 | 5.06 | 4.92 | 4.89 |
| 10 | 9.54 | 9.01 | 9.87 | 10.38 | 10.08 | 10.14 |
| 30 | 27.74 | 27.57 | 28.83 | 30.37 | 30.66 | 30.31 |

TABLE 6

| | | Example 4 | Example 4 | Example 4 | Example 4 |
|---|---|---|---|---|---|
| | | cyclized | cyclized | cyclized | cyclized |
| | Example 3 | CRP2 | CRP2 | CRP2 | CRP2 |
| | recombinant | day 1 of | day 3 of | day 6 of | day 8 of |
| | CRP2 | heating | heating | heating | heating |
| N-terminal | 40% | 42% | 54% | 67% | 78% |
| cyclization | | | | | |
| rate | | | | | |
| Theoretical | | | | | |
| value | | | | | |
| (mg/dL) | Relative value (recombinant CRP/natural human CRP) | | | | |
| 1 | 103% | 100% | 102% | 104% | 102% |
| 5 | 98% | 96% | 100% | 104% | 101% |
| 10 | 94% | 89% | 97% | 102% | 99% |
| 30 | 91% | 91% | 95% | 100% | 101% |

TABLE 7

| | | Example 4 | Example 4 | Example 4 | Example 4 | |
|---|---|---|---|---|---|---|
| | | cyclized | cyclized | cyclized | cyclized | |
| | Example 3 | CRP2 | CRP2 | CRP2 | CRP2 | Natural |
| | recombinant | day 1 of | day 3 of | day 6 of | day 8 of | human |
| | CRP2 | heating | heating | heating | heating | CRP |
| N-terminal | 40% | 42% | 54% | 67% | 78% | 95% |
| cyclization rate | | | | | | |
| Theoretical | Measured value (mg/dL) | | | | | |
| value (mg/dL) | | | | | | |
| 1 | 0.79 | 0.76 | 0.77 | 0.79 | 0.78 | 0.76 |
| 5 | 4.80 | 4.69 | 4.88 | 5.06 | 4.92 | 4.89 |
| 10 | 9.54 | 9.01 | 9.87 | 10.38 | 10.08 | 10.14 |
| 30 | 27.74 | 27.57 | 28.83 | 30.37 | 30.66 | 30.31 |

### INDUSTRIAL APPLICABILITY

The CRPs of the present invention are particularly useful in the medical and diagnostic fields as diagnostic raw materials for use in latex reagents with excellent reactivity in a high CRP concentration range.

1. Recombinant C-reactive proteins produced by genetic recombination, 55% or more of the C-reactive proteins having a pyroglutamylated N-terminal.

2. The recombinant C-reactive proteins according to claim 1, wherein 65% or more of the C-reactive proteins have a pyroglutamylated N-terminal.

3. The recombinant C-reactive proteins according to claim 1, wherein 75% or more of the C-reactive proteins have a pyroglutamylated N-terminal.

4. The recombinant C-reactive proteins according to claim 1, wherein 85% or more of the C-reactive proteins have a pyroglutamylated N-terminal.

5. The recombinant C-reactive proteins according to claim 1, wherein the recombinant C-reactive proteins are bacterial recombinant proteins.

6. The recombinant C-reactive proteins according to claim 5, wherein the bacterium is Escherichia coli.

7. The recombinant C-reactive proteins according to claim 1, wherein the C-reactive proteins are derived from a human.

8. The recombinant C-reactive proteins according to claim 1, wherein the C-reactive proteins comprise any of the following polypeptides (a) to (c):
(a) a polypeptide represented by SEQ ID No: 1 or SEQ ID No: 2;
(b) a polypeptide comprising an amino acid sequence including substitution, deletion, insertion, and/or addition of one or more amino acid residues in the amino acid sequence represented by SEQ ID No: 1 or SEQ ID No: 2, and having antigenicity against anti-C-reactive protein antibody; and
(c) a polypeptide comprising an amino acid sequence having 90% or more identity to the amino acid sequence represented by SEQ ID No: 1 or SEQ ID No: 2, and having antigenicity against anti-C-reactive protein antibody.

9. A calibrator comprising the recombinant C-reactive proteins according to claim 1.

10. Control serum comprising the recombinant C-reactive proteins according to claim 1.

11. A method for quantifying C-reactive proteins in a sample using the calibrator comprising the recombinant C-reactive proteins according to claim 9.

12. A method for quantifying C-reactive proteins in a sample using the control serum comprising the recombinant C-reactive proteins according to claim 10.

13. The method for quantifying C-reactive proteins in a sample according to claim 11, by latex turbidimetric immunoassay using latex particles on which anti-C-reactive protein antibody is immobilized.

14. The method for quantifying C-reactive proteins in a sample according to claim 12, by latex turbidimetric immunoassay using latex particles on which anti-C-reactive protein antibody is immobilized.
